(19) United States
(12) Patent Application Publication
(10) Pub. No.: US 2006/0188590 A1
(43) Pub. Date: Aug. 24, 2006

(54) Compositions for treating diabetes or obesity

(76) Inventor: Mitsunori Ono, Lexington, MA (US)

Correspondence Address: FISH & RICHARDSON PC, P.O. BOX 1022, MINNEAPOLIS, MN, 55440-1022, US

(21) Appl. No.: 11/326,102

(22) Filed: Jan. 4, 2006

Related U.S. Application Data
(60) Provisional application No. 60/641,642, filed on Jan. 5, 2005.

(51) Int. Cl. A61K 36/906; A61K 36/185; A61K 31/7048; A61K 31/353; A61K 31/522; A61K 36/63

(52) U.S. Cl. 424/756; 424/778; 514/27; 514/263.31; 514/456; 424/769; 514/343; 514/553; 514/561; 514/690; 514/733

(57) ABSTRACT

This invention relates to a composition that includes two compounds selected from a group of nine members, i.e., an α-glucosidase inhibitor, an intestinal glucose transporter inhibitor, a glycation inhibitor, a nitric oxide production inhibitor, an aldose reductase inhibitor, a PPAR agonist, an adipocytokine activator, a glucose uptake enhancer, and a thermogenesis enhancer, in which the two compounds are two different members; and each compound is naturally occurring in a plant and is provided in the form of a plant extract. This invention also relates to a method of treating diabetes or obesity with the above-mentioned composition.

### CROSS REFERENCE TO RELATED APPLICATION

Under 35 U.S.C. § 119, this application claims priority to U.S. Provisional Application Ser. No. 60/641,642, filed Jan. 5, 2005, the contents of which are incorporated herein by reference.

### BACKGROUND

Treating a disease with two or more drugs can be advantageous when the combination of the drugs results in synergistic effect. Indeed, combination therapy has been used extensively in treating diseases such as HIV and cancers. However, many patients treated with combination therapy suffer serious adverse effect in comparison with those treated with single therapy. As a result, this approach is generally used only in treating life threatening diseases for which no better therapy is available.

It is desirable to develop combination therapy that has improved efficacy and little or no side effect for use in treating non-life threatening diseases or disorders, such as diabetes or obesity.

### SUMMARY

This invention relates to a composition containing two or more naturally-occurring compounds that is effective in treating diabetes or obesity.

In one aspect, this invention features a composition that includes two different compounds selected from a group of nine members, i.e., an α-glucosidase inhibitor, an intestinal glucose transporter inhibitor, a glycation inhibitor, a nitric oxide production inhibitor, an aldose reductase inhibitor, a peroxisome proliferator-activated receptor (“PPAR”) agonist, an adipocytokine activator, a glucose uptake enhancer, and a thermogenesis enhancer, in which the two compounds are two different members. Preferred members are an α-glucosidase inhibitor, an intestinal glucose transporter inhibitor, a nitric oxide production inhibitor, an adipocytokine activator, and a glucose uptake enhancer. The composition can also contain a third compound, a fourth compound, or a fifth compound. Each of these three compounds, different from each other and from the above-mentioned two compounds, can be selected from the group mentioned above and all compounds can be different members. Each compound in the composition can be naturally occurring and conveniently provided in the form of a plant extract. The plant extract can be either a pure compound or a mixture, as long as it is obtained from a plant.

Examples of an α-glucosidase inhibitor include salacinol, anthocyanin (including, e.g., cyanidin or cyanidin-3-glucoside), isoflavone (including, e.g., genistein, daidzein, and glycitein), and luteolin. Examples of an intestinal glucose transporter inhibitor include rutin, isoflavone, equol, isoquercitrin, quercetin, spiraeosid, and catechin (including, e.g., (+)-catechin, epigallocatechin gallate, epicatechin, epicatechin gallate, epigallocatechin). Examples of a glycation inhibitor include glycine, taurine, and rutin. Examples of a nitric oxide production inhibitor include procyanidin, proanthocyanidin, quercetin, rutin, morin, apigenin, hesperetin, naringin, sesamol, chlorogenic acid, catechin, ellagic acid, caffeic acid, isoflavone, zerumbone, curcumin, and resveratrol. Examples of an aldose reductase inhibitor include isoquercitrin, quercitrin, guaijaverin, desmanthin-1,8-hydroxydaidzein, or isoaffinettin. Examples of a PPAR agonist include isoflavone, and hops alpha acid (including, e.g., isohumulone, and isocohumulone). Examples of an adipocytokine activator include anthocyanin and xanthohumol. Examples of a glucose uptake enhancer include procyanidin, proanthocyanidin, quercetin, rutin, a-lipoic acid, myricetin, and epicatechin. Examples of a thermogenesis enhancer include saponin, capsaicin, gingerol, catechin, caffeine, nicotine, and an extract of olive oil.

The compounds mentioned above include both the compounds themselves and their derivatives (e.g., sugar derivatives). For example, quercetin derivatives include quercetin-3-O-glucoside, quercetin-5-O-glucoside, quercetin-7-O-glucoside, quercetin-9-O-glucoside, quercetin-3-O-rutinoside, quercetin-3-O-[α-rhamnosyl-(1→2)-α-rhamnosyl-(1→6)]-β-glucoside, quercetin-3-O-galactoside, quercetin-7-O-galactoside, quercetin-3-O-rhamnoside, and quercetin-7-O-galactoside. Examples of daidzein derivatives include 6″-O-acetyldaidzin and 6″-O-malonyldaidzin. Examples of genistein derivatives include 6″-O-acetylgenistin and 6″-O-malonylgenistin. Examples of glycitein derivatives include 6″-O-acetylglycitin and 6″-O-malonylglycitin.

Referring to the above-mentioned nine members, certain compounds in the composition of this invention can possess the biological properties of two or more of the nine members. For example, anothcyanin inhibits α-glucosidase and activates adipocytokine at the same time. Of note, two compounds are two different members when one compound has at least one biological property different from the other compound. Thus, anothcyanin (which inhibits α-glucosidase and activates adipocytokine) is a member different from salacinol (which only inhibits α-glucosidase). Also, each of the nine members can be a single compound or a family of compounds (e.g., isoflavone, procyanidin, catechin, proanthocyanin, or anythocyanin). Two or more species of the same family of compounds are considered as one member. Take isoflavone for example. It includes a number of species, such as genistein, daidzein, and glycitein, which are considered as one member.

In another aspect, this invention features a method of treating diabetes or obesity. The method includes administering to a subject in need thereof an effective amount of any of the compositions described above.

Also within the scope of this invention is a composition described above for use in treating diabetes or obesity, and the use of such a composition for the manufacture of a medicament for the just-mentioned treatment.

The details of one or more embodiments of the invention are set forth in the description below. Other features, objects, and advantages of the invention will be apparent from the description and from the claims.

### DETAILED DESCRIPTION

A composition of this invention includes two or more compounds selected from the group of nine members consisting of an α-glucosidase inhibitor, an intestinal glucose transporter inhibitor, a glycation inhibitor, a nitric oxide production inhibitor, an aldose reductase inhibitor, a PPAR agonist, an adipocytokine activator, a glucose uptake enhancer, and a thermogenesis enhancer. The composition affects two or more of the following events that take place during carbohydrate metabolism in a human body: (1) hydrolysis of starch into monosaccharides, such as glucose, (2) entrance of glucose into the blood from small intestine, (3) reactions between glucose and proteins or nucleic acids in the blood, (4) binding of insulin to its receptors, (5) uptake of glucose into cells from the blood, and (6) oxidation of glucose in cells.

An α-glucosidase inhibitor affects event (1). Specifically, it suppresses the activity of α-glucosidase, thereby reducing the hydrolysis of starch into oligosaccharides (lactose) or monosaccharides (e.g., glucose, fructose, or galactose).

An intestinal glucose transporter inhibitor affects event (2). It reduces transport of monosaccharides from small intestine to the blood through glucose transporters located in cell membranes. The term “glucose transporter” refers to both glucose transporters and glucose co-transporters. Examples include glucose transporter-1 to glucose transporter-12, proton myo-inositol transporter, and sodium-dependent glucose cotransporter-1 to sodium-dependent glucose cotransporter-6.

A glycation inhibitor, an aldose reductase inhibitor, and a nitric oxide production inhibitor affect event (3) by reducing the side effects generated by the high concentrations of glucose in the blood. The term “glycation inhibitor” refers to non-enzymatic glycation inhibitors. Specifically, proteins and nucleic acids in the blood undergo glycosylation reactions in the absence of any enzyme. These reactions alter the structures and functions of the proteins and nucleic acids, thereby causing certain disorders. A high glucose concentration in the blood drives the glycosylation reactions. The glycation inhibitor suppresses non-enzymatic glycosylation reactions and therefore prevents certain diabetic disorders. An aldose reductase inhibitor prevents or reduces the action of aldose reductase. Aldose reductase is an enzyme present in the eye and many other parts of the body. It promotes conversion of glucose into sorbitol. Diabetic patients can have high concentrations of sorbitol in eye cells and nerve cells, which lead to retinopathy and neuropathy, respectively. An aldose reductase inhibitor reduces the formation of sorbitol, thereby preventing or delaying these complications of diabetes. A nitric oxide production inhibitor reduces excess production of inducible nitric oxide resulting from diabetes, which can cause diabetic complications and insulin resistance.

A PPAR agonist and an adipocytokine activator affect event (4) by enhancing the insulin action and ameliorating the insulin resistance. PPAR receptors belong to a family of nuclear receptors that regulate lipid metabolism. A PPAR agonist binds to PPAR receptors, thereby improving muscle insulin action and increasing insulin sensitivity. An adipocytokine enhancer improves adipocytokin secretion and up-regulates the adipocyte specific gene, thereby improving insulin sensitivity and glucose tolerance.

A glucose uptake enhancer affects event (5) by facilitating uptake of glucose from the blood to the cells, in which glucose is oxidized to generate energy.

A thermogenesis activator affects event (6) by promoting oxidation metabolism of glucose (e.g., in TCA cycles) in the cells.

The compounds in the composition of this invention can be obtained by any suitable means. They can be obtained from commercial sources or, preferably, from various plants. Set forth below are a number of examples. Rutin can be provided in an extract of buckwheat. Quercetin, morin, and myricetin can be provided in an extract of onion, grape seed, or berries. Catechin can be provided in an extract of green tea or black tea. Anthocyanin can be provided in an extract of red clover, acai, and cherries. Hesperitin and naringin can be provided in an extract of citrus fruits or licorice. Xanthohumol, isohumulone, isocohumulone, α-acid can be provided in an extract of hops. Procyanidin can be provided in an extract of cocoa, grape seed, hops, or pine bark. Isoflavone (such as genistein and daidzein) can be provided in an extract of soy germ, soy bean, or red clover. Chlorogenic acid and caffeic acid can be provided in an extract of coffee bean. Apigenin and luteolin can be provided in an extract of celery, parsley, red pepper, or mint. Isoquercitrin and quercitrin can be provided in an extract of onion or seed pods of fava d'anta. Sesamol can be provided in an extract of sesame seed. Curcumin can be provided in an extract of turmeric. Resveratrol can be provided in an extract of red grape skin. Ellagic acid can be provided in an extract of berries. Gingerol can be provided in an extract of ginger root. Capsaicin can be provided in an extract of cayenne.

Each of the extracts mentioned above can be prepared by first immersing a pulverized plant (or a part of a plant) in an aqueous solvent, an organic solvent, or a mixture of solvents. Examples of a suitable organic solvent include ethanol, dichloromethane, or hexane. The crude extract thus obtained can be filtered or centrifuged to remove any insoluble materials. A purified extract can then be obtained from the crude extract using liquid chromatography (e.g., high-pressure liquid chromatography) or other suitable methods. An extract can be produced either by a batch method or by a continuous method.

The composition of this invention can be a dietary supplement or a pharmaceutical formulation. As a dietary supplement, additional nutrients, such as minerals or amino acids may be included. The composition can also be a drink or a food product, e.g., tea, soft drink, juice, milk, coffee, cookie, cereal, chocolate, and snack bar.

The composition of this invention can be in the form of a solution. For example, it can be an aqueous solution optionally containing a non-aqueous co-solvent, such as an alcohol. The composition can also be in the form of powder, paste, jelly, capsule, or tablet. Lactose and corn starch are commonly used as diluents for capsules and as carriers for tablets. Lubricating agents, such as magnesium stearate, are typically added to form tablets.

The composition of this invention can be sweetened, if necessary, by adding a sweetener such as sorbitol, maltitol, hydrogenated glucose syrup and hydrogenated starch hydrolyzate, high fructose corn syrup, cane sugar, beet sugar, pectin, or sucralose.

The composition, in any of the forms described above, can be used for treating diabetes or obesity. It can be used to treat an obese patient who has no diabetes by, e.g., preventing production of glucose or promoting the oxidation of glucose. It can also be used to treat a diabetic patient who is not obese by, e.g., preventing glucose from entering into the blood. The term “treating” refers to the administration of an effective amount of a composition of this invention to a subject, who has diabetes or obesity, or a symptom or a predisposition of such a disease, with the purpose to cure, alleviate, relieve, remedy, or ameliorate diabetes or obesity, the symptoms of it, or the predispositions towards it. The term “administration” covers oral or parenteral delivery to a subject a composition of this invention in any suitable form, e.g., food product, beverage, tablet, and capsule. The term “parenteral” refers to subcutaneous, intracutaneous, intravenous, intramuscular, intraarticular, intraarterial, intrasynovial, intrasternal, intrathecal, intralesional, and intracranial injection, as well as various infusion techniques. The term “effective amount” refers to a dose of the composition that is sufficient to provide a therapeutic benefit on a subject. Both in vivo and in vitro studies can be conducted to determine optimal administration routes and doses.

Without further elaboration, it is believed that the above description has adequately enabled the present invention. The following specific examples are, therefore, to be construed as merely illustrative, and not limitative of the remainder of the disclosure in any way whatsoever.

### EXAMPLE 1

Formulation 1 was prepared as follows. To 595 ml purified water were added: taurine (2000 mg, 99.8% human grade), rutin (500 mg, 99.8% human grade), a grape seed extract (600 mg; containing 300 mg procyanidin), a soy extract (1670 mg; containing 100 mg genistein), a bilberry extract (800 mg; containing 200 mg of anthocyanin), and sucralose (4 g; Splendag) at room temperature. All ingredients can be obtained from Sigma, St. Louis, Mo.; Equine Product Inc, San Juan Capistrano, Calif.; Aldrich, Milwaukee, Wis.; or Cargill Health & Food Technologies, Wayzata, Minn. The above mixture was vigorously stirred by using a food mixer and then diluted up to 660 ml (21 oz) with orange juice concentrates. Typically, a patient takes 330 ml of Formulation 1 twice a day.

### EXAMPLE 2

Formulation 2 was prepared as follows. A bilberry extract (800 mg; containing 200 mg of anthocyanin), glycine (200 mg, 99.8% human grade), alpha lipoic acid (200 mg, 99.8% ), a soy extract (560 mg; containing 100 mg daizein), and thea-flan 90S (600 mg; containing 300 mg of epigallocatechin gallate) were vigorously mixed and capsulated into four gelatin capsules. All ingredients can be obtained from Sigma, St. Louis, Mo.; Equine Product Inc, San Juan Capistrano, Calif,; Aldrich, Milwaukee, Wis.; Cargill Health & Food Technologies, Wayzata, Minn., or ITOEN (North America), Brooklyn, N.Y. Typically, a patient takes two capsules of Formulation 2 twice a day.

### EXAMPLE 3

Formulation 3 was prepared as follows. A purified powdered extract from the seeds and pods of the brazilian shrub “fava d'anta” (Dimorphandra mollis) (600 mg; containing 300 mg of isoquercitrin), resveratrol (200 mg, 99.8%), a soy extract (560 mg; containing 100 mg daizein), a bilberry extract (800 mg; containing 200 mg of anthocyanin), thea-flan 90S (600 mg; containing 300 mg of epigallocatechin gallate) were vigorously mixed and capsulated into six gelatin capsules. All ingredients can be obtained from Sigma, St. Louis, Mo.; Equine Product Inc, San Juan Capistrano, Calif,; Aldrich, Milwaukee, Wis.; Cargill Health & Food Technologies, Wayzata, Minn.; or ITOEN (North America), Brooklyn, N.Y. Typically, a patient takes two capsules of Formulation 3 three times a day.

### EXAMPLE 4

Formulation 4 was prepared as follows. A Brazilian acai berry extract (300 mg; containing 200 mg anthocyanin), alpha acids from hops (500 mg, 98%), alpha lipoic acid (200 mg, 99.8%), a pine bark extract (471 mg; containing 400 mg of proanthocyanidin) were vigorously mixed and capsulated into three gelatin capsules. All ingredients can be obtained from Sigma, St. Louis, Mo.; Equine Product Inc, San Juan Capistrano, Calif,; Aldrich, Milwaukee, Wis.; AMAX Nutraceticals, City of Industry, Calif,; and John I Haas, Washington D.C. Typically, a patient takes one capsule of Formulation 4 three times a day.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Furthermore, from the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A composition, comprising a first compound and a second compound each selected from the group of nine members consisting of an α-glucosidase inhibitor, an intestinal glucose transporter inhibitor, a glycation inhibitor, a nitric oxide production inhibitor, an aldose reductase inhibitor, a PPAR agonist, an adipocytokine activator, a glucose uptake enhancer, and a thermogenesis enhancer; wherein the first compound and the second compound are two different members, and each compound is naturally occurring in a plant.

2. The composition of claim 1, wherein each compound is provided in the form of a plant extract.

3. The composition of claim 1, wherein the first compound or the second compound is anthocyanin, procyanidin, or proanthocyanidin.

4. The composition of claim 1, further comprising a third compound selected from the group of nine members consisting of an α-glucosidase inhibitor, an intestinal glucose transporter inhibitor, a glycation inhibitor, a nitric oxide production inhibitor, an aldose reductase inhibitor, a PPAR agonist, an adipocytokine activator, a glucose uptake enhancer, and a thermogenesis enhancer; wherein the first, second, and third compounds are three different members, and the third compound is naturally occurring in a plant.

5. The composition of claim 4, wherein each compound is provided in the form of a plant extract.

6. The composition of claim 4, wherein the first compound or the second compound is anthocyanin, procyanidin, or proanthocyanidin.

7. The composition of claim 4, wherein the first, second, and third compounds are selected from the group of five members consisting of an α-glucosidase inhibitor, an intestinal glucose transporter inhibitor, a nitric oxide production inhibitor, an adipocytokine activator, and a glucose uptake enhancer.

8. The composition of claim 7, wherein
the α-glucosidase inhibitor is salacinol, anthocyanin, isoflavone, or luteolin;
the intestinal glucose transporter inhibitor is rutin, isoflavone, equol, isoquercitrin, quercetin, spiraeosid, or catechin;
the nitric oxide production inhibitor is procyanidin, proanthocyanidin, quercetin, rutin, morin, apigenin, hesperetin, naringin, sesamol, chlorogenic acid, catechin, ellagic acid, caffeic acid, isoflavone, zerumbone, curcumin, or resveratrol;
the adipocytokine activator is anthocyanin or xanthohumol; and
the glucose uptake enhancer is procyanidin, proanthocyanindin, quercetin, rutin, α-lipoic acid, myricetin, or epicatechin.

9. The composition of claim 8, wherein the α-glucosidase inhibitor is anthocyanin or isoflavone.

10. The composition of claim 8, wherein the intestinal glucose transporter inhibitor is rutin, isoquercetrin, catechin, or quercetin.

11. The composition of claim 8, wherein the nitric oxide production inhibitor is procyanidin, proanthocyanidin, curcumin, catechin, resveratrol, or rutin.

12. The composition of claim 8, wherein the glucose uptake enhancer is procyanidin, proanthocyanidin, α-lipoic acid, or epicatechin.

13. The composition of claim 7, further comprising a fourth compound selected from the group of five members consisting of an α-glucosidase inhibitor, an intestinal glucose transporter inhibitor, a nitric oxide production inhibitor, an adipocytokine activator, and a glucose uptake enhancer, wherein the first, second, third, and fourth compounds are four different members, and the fourth compound is naturally occurring in a plant.

14. The composition of claim 13, wherein each compound is provided in the form of a plant extract.

15. The composition of claim 13, wherein the first compound or the second compound is anthocyanin, procyanidin, or proanthocyanidin.

16. The composition of claim 13, further comprising a fifth compound selected from the group of five members consisting of an α-glucosidase inhibitor, an intestinal glucose transporter inhibitor, a nitric oxide production inhibitor, an adipocytokine activator, and a glucose uptake enhancer, wherein the first, second, third, fourth, and fifth compounds are five different members, and the fifth compound is naturally occurring in a plant.

17. The composition of claim 16, wherein each compound is provided in the form of a plant extract.

18. The composition of claim 16, wherein the first compound or the second compound is anthocyanin, procyanidin, or proanthocyanidin.

19. The composition of claim 4, wherein the α-glucosidase inhibitor is salacinol, anthocyanin, isoflavone, or luteolin;
the intestinal glucose transporter inhibitor is rutin, isoflavone, equol, isoquercitrin, quercetin, spiraeosid, or catechin;
the glycation inhibitor is glycine, taurine, or rutin;
the nitric oxide production inhibitor is procyanidin, proanthocyanidin, quercetin, rutin, morin, apigenin, hesperetin, naringin, sesamol, chlorogenic acid, catechin, ellagic acid, caffeic acid, isoflavone, zerumbone, curcumin, or resveratrol;
the aldose reductase inhibitor is isoquercitrin, quercitrin, guaijaverin, desmanthin-1,8-hydroxydaidzein, or isoaffinettin;
the PPAR agonist is isoflavone, or hops alpha acid;
the adipocytokine activator is anthocyanin or xanthohumol;
the glucose uptake enhancer is procyanidin, proanthocyanidin, quercetin, rutin, α-lipoic acid, myricetin, or epicatechin; and
the thermogenesis enhancer is saponin, capsaicin, gingerol, catechin, caffeine, nicotine, or an olive oil extract.

20. The composition of claim 19, wherein the α-glucosidase inhibitor is anthocyanin or isoflavone.

21. The composition of claim 19, wherein the intestinal glucose transporter inhibitor is rutin, isoquercetrin, catechin, or quercetin.

22. The composition of claim 19, wherein the glycation inhibitor is glycine or taurine.

23. The composition of claim 19, wherein the nitric oxide production inhibitor is procyanidin, proanthocyanidin, curcumin, catechin, resveratrol, or rutin.

24. The composition of claim 19, wherein the aldose reductase inhibitor is isoquercitrin or quercitrin.

25. The composition of claim 19, wherein the PPAR agonist is isoflavone or isohumulone.

26. The composition of claim 19, wherein the glucose uptake enhancer is procyanidin, (x-lipoic acid, or epicatechin.

27. The composition of claim 19, wherein the thermogenesis enhancer is catechin, caffeine, capsaicin, or gingerol.

28. The composition of claim 1, wherein the first and second compounds are selected from the group of five members consisting of an α-glucosidase inhibitor, an intestinal glucose transporter inhibitor, a nitric oxide production inhibitor, an adipocytokine activator, and a glucose uptake enhancer.

29. The composition of claim 28, wherein the first compound or the second compound is anthocyanin, procyanidin, or proanthocyanidin.
